(19) United States
(12) Patent Application Publication
(10) Pub. No.: US 2015/0289894 A1
(43) Pub. Date: Oct. 15, 2015

(54) FLUID EJECTION DEVICE

(71) Applicant: SEIKO EPSON CORPORATION, Tokyo (JP)

(72) Inventors: Koichiro MIYAZAKI, Ina-shi (JP); Masaki GOMI, Hino-shi (JP)

(21) Appl. No.: 14/682,801

(22) Filed: Apr. 9, 2015

(30) Foreign Application Priority Data
Apr. 10, 2014 (JP) 2014-080819

(51) Int. Cl. A61B 17/3203

(52) U.S. Cl. CPC A61B 17/3203; A61B 2017/00154

(57) ABSTRACT

A fluid ejection device includes first and second fluid ejection units that eject fluid in a pulse-like manner according to first and second drive signals, respectively. A first fluid supplying unit supplies fluid to the first fluid ejection unit via a first channel. A second fluid supplying unit supplies fluid to the second fluid ejection unit via a second channel. A first fluid-ejecting control unit outputs the first drive signal after changing the fluid in the first channel to a predetermined state. A second fluid-ejecting control unit outputs the second drive signal after changing the fluid in the second channel to a predetermined state. A malfunction detecting unit detects malfunctions that might occur in the first fluid ejection unit, fluid supplying unit, channel, or fluid-ejecting control unit. When the malfunction is detected, the second fluid-ejecting control unit starts the second preparation operation.

This application claims the benefit of Japanese Patent Application No. 2014-080819, filed on Apr. 10, 2014. The content of the aforementioned application is incorporated by reference in its entirety.

### BACKGROUND

1. Technical Field

The present invention relates to a fluid ejection device.

2. Related Art

There is known a technique for ejecting fluid in a pulse-like manner to perform incision, excision, or the like of a target object. For example, in the medical field, as a surgical instrument for incising or excising a biological tissue, there is proposed a fluid ejection device including a plurality of pulsed flow generating units having different ejecting characteristics according to characteristics of target regions to which fluid is ejected, a fluid supplying unit that supplies the fluid to the plurality of pulsed flow generating units, and a fluid supply path from the fluid supply unit to the pulsed flow generating units (see, for example, JP-A-2010-053766 (Patent Literature 1)).

Such a fluid ejection device is manufactured with extremely high reliability. However, in preparation for an accidental failure, a further improvement of reliability can be attained by imparting redundancy to components of the fluid ejection device. In this case, even if malfunction occurs in one component during a surgical operation, it is possible to continue the surgical operation using an auxiliary component.

However, it is not enough for the improvement of reliability to simply prepare the auxiliary components of the fluid ejection device. It is necessary to perform beforehand preparatory work such as various settings, adjustments, and the like for the auxiliary components as well.

Therefore, when redundancy is imparted to the components of the fluid ejection device, it is necessary to prepare the auxiliary components, which are less likely to be actually used in the surgical operation, to be used in the surgical operation at any time. Therefore, the preparation work is necessary and excess electric power is consumed.

Therefore, there is a demand for a technique for reducing wastes that occur when redundancy is imparted to the components of the fluid ejection device.

### SUMMARY

A fluid ejection device according to an aspect of the invention includes: a first fluid ejection unit configured to eject fluid in a pulse-like manner according to a first drive signal; a second fluid ejection unit configured to eject the fluid in a pulse-like manner according to a second drive signal; a first fluid supplying unit configured to supply the fluid to the first fluid ejection unit via a first channel; a second fluid supplying unit configured to supply the fluid to the second fluid ejection unit via a second channel; a first fluid-ejecting control unit configured to execute a first preparation operation for changing the fluid in the first channel to a predetermined state and, after the first preparation operation is completed, output the first drive signal to the first fluid ejection unit; a second fluid-ejecting control unit configured to execute a second preparation operation for changing the fluid in the second channel to a predetermined state and, after the second preparation operation is completed, output the second drive signal to the second fluid ejection unit; and an malfunction detecting unit configured to detect malfunction that occurs in at least any one of the first fluid ejection unit, the first fluid supplying unit, the first channel, and the first fluid-ejecting control unit. When the malfunction detecting unit detects the malfunction, the second fluid-ejecting control unit starts the second preparation operation.

Other features of the invention will be made apparent by the description of this specification and the accompanying drawings.

### BRIEF DESCRIPTION OF THE DRAWINGS

The invention will be described with reference to the accompanying drawings, wherein like numbers reference like elements.

FIG. 1 is a block diagram showing an example of the overall configuration of a fluid ejection device according to an embodiment of the invention.

FIG. 2 is a block diagram showing the configuration of a pump according to the embodiment of the invention.

FIG. 3 is a block diagram showing the configuration of the pump according to the embodiment of the invention.

FIG. 4 is a sectional view showing the structure of a pulsation generator according to the embodiment of the invention.

FIG. 5 is a plan view showing a form of an inlet channel according to the embodiment of the invention.

FIG. 6 is a block diagram showing the configuration of a first pump control unit and a second pump control unit according to the embodiment of the invention.

FIG. 7 is a flowchart for explaining a flow of processing of the first pump control unit according to the embodiment of the invention.

FIG. 8 is a flowchart for explaining a flow of processing of the second pump control unit according to the embodiment of the invention.

### DESCRIPTION OF EXEMPLARY EMBODIMENTS

### Overview

At least matters described below are made apparent by the description of this specification and the drawings.

A fluid ejection device includes: a first fluid ejection unit configured to eject fluid in a pulse-like manner according to a first drive signal; a second fluid ejection unit configured to eject the fluid in a pulse-like manner according to a second drive signal; a first fluid supplying unit configured to supply the fluid to the first fluid ejection unit via a first channel; a second fluid supplying unit configured to supply the fluid to the second fluid ejection unit via a second channel; a first fluid-ejecting control unit configured to execute a first preparation operation for changing the fluid in the first channel to a predetermined state and, after the first preparation operation is completed, output the first drive signal to the first fluid ejection unit; a second fluid-ejecting control unit configured to execute a second preparation operation for changing the fluid in the second channel to a predetermined state and, after the second preparation operation is completed, output the second drive signal to the second fluid ejection unit; and an malfunction detecting unit configured to detect malfunction that occurs in at least any one of the first fluid ejection unit, the first fluid supplying unit, the first channel, and the first fluid-ejecting control unit. When the malfunction detecting unit detects the malfunction, the second fluid-ejecting control unit starts the second preparation operation.

With such a fluid ejection device, it is possible to reduce wastes that occur when redundancy is imparted to the components of the fluid ejection device.

It is preferable that the malfunction detecting unit determines, when detecting the malfunction, according to content of the detected malfunction, whether the ejecting of the fluid by the first fluid ejection unit can be continued, and the second fluid ejecting-control unit starts the second preparation operation even if it is determined that the ejecting of the fluid by the first fluid ejection unit can be continued.

With such a fluid ejection device, it is possible to quickly start the second preparation operation while the ejecting of the fluid by the first fluid ejection unit is possible. Therefore, it is possible to improve possibility that the ejecting of the fluid by the second fluid ejection unit can be started before the ejecting of the fluid by the first fluid ejection unit is disabled.

It is preferable that, when determining that the ejecting of the fluid by the first fluid ejection unit cannot be continued, the malfunction detecting unit outputs predetermined notification information to that effect.

With such a fluid ejection device, it is possible to quickly inform an operator such as a surgeon that the ejecting from the first fluid ejection unit has been disabled. It is possible to further improve the safety of the fluid ejection device.

It is preferable that, when the second preparation operation is completed, the second fluid-ejecting control unit outputs predetermined notification information to that effect.

With such a fluid ejection device, it is possible to quickly inform the operator such as the surgeon that the ejecting from the second fluid ejection unit has been enabled. It is possible to quickly switch the first fluid ejection unit to the second fluid ejection unit to eject the fluid.

It is preferable that the first preparation operation includes an operation for causing the fluid to reach the first fluid ejection unit from the first fluid supplying unit via the first channel, and the second preparation operation includes an operation for causing the fluid to reach the second fluid ejection unit from the second fluid supplying unit via the second channel.

With such a fluid ejection device, at a point when the ejecting is started using the first fluid ejection unit or the second fluid ejection unit, the fluid already reaches the first fluid ejection unit or the second fluid ejection unit. Therefore, it is possible to immediately start the ejecting of the fluid.

It is preferable that the fluid ejection device further includes: a first pressure detecting unit configured to detect pressure of the fluid in the first channel; and a second pressure detecting unit configured to detect pressure of the fluid in the second channel, the first preparation operation further includes an operation for causing the first fluid supplying unit to increase the pressure of the fluid in the first channel to a first predetermined value or more, and the second preparation operation further includes an operation for causing the second fluid supplying unit to increase the pressure of the fluid in the second channel to a second predetermined value or more.

With such a fluid ejection device, it is possible to eject the fluid with stable strength from a point in time of the start of the ejecting.

It is preferable that the first preparation operation further includes an operation for causing the first fluid supplying unit and the first fluid ejection unit to remove air bubbles included in the fluid in the first channel, and the second preparation operation further includes an operation for causing the second fluid supplying unit and the second fluid ejection unit to remove air bubbles included in the fluid in the second channel.

With such a fluid ejection device, it is possible to stably eject the fluid in a pulse-like manner without the air bubbles from being discharged together with the fluid after the start of the ejecting.

It is preferable that the operation for causing the first fluid supplying unit and the first fluid ejection unit to remove the air bubbles included in the fluid in the first channel includes an operation for causing the first fluid ejection unit to eject the fluid in a pulse-like manner in a state in which the first fluid supplying unit is caused to increase a flow rate of the fluid flowing in the first channel in a predetermined time to a predetermined amount, and the operation for causing the second fluid supplying unit and the second fluid ejection unit to remove the air bubbles included in the fluid in the second channel includes an operation for causing the second fluid ejection unit to eject the fluid in a pulse-like manner in a state in which the second fluid supplying unit is caused to increase a flow rate of the fluid flowing in the second channel in a predetermined time to a predetermined amount.

With such a fluid ejection device, it is possible to effectively remove the air bubbles.

It is preferable that the fluid ejection device further includes: a first pressure detecting unit configured to detect pressure of the fluid in the first channel; and a second pressure detecting unit configured to detect pressure of the fluid in the second channel, the first preparation operation includes only an operation for causing the first fluid supply unit to increase the pressure of the fluid in the first channel to a first predetermined value or more, and the second preparation operation includes only an operation for causing the second fluid supplying unit to increase the pressure of the fluid in the second channel to a second predetermined value or more.

With such a fluid ejection device, it is possible to eject the fluid with stable strength from a point in time of the start of the ejecting. When the fluid already reaches the first fluid ejection unit or the second fluid ejection unit, it is possible to prevent an unnecessary preparation operation from being wastefully performed. It is possible to reduce time required for the preparation operation.

It is preferable that, when detecting the malfunction, the malfunction detecting unit outputs predetermined notification information to that effect.

With such a fluid ejection device, it is possible to quickly inform the operator such as the surgeon that the malfunction has occurred in at least any one of the first fluid ejection unit, the first fluid supplying unit, the first channel, and the first fluid-ejecting control unit. It is possible to further improve the safety of the fluid ejection device.

### Overall Configuration

An embodiment of the invention is explained below with reference to the drawings. A fluid ejection device according to this embodiment is adoptable for cleaning, cutting, and the like of fine objects, structures, biological tissues, and the like. In the embodiment explained below, a fluid ejection device suitable for a surgical knife for incising or excising a biological tissue is illustrated. Therefore, fluid used in the fluid ejection device according to this embodiment is water, saline, predetermined chemical, or the like. Note that, drawings referred to in the following explanation are schematic diagrams in which, for convenience of illustration, longitudinal and lateral scales of members and portions are different from actual scales.

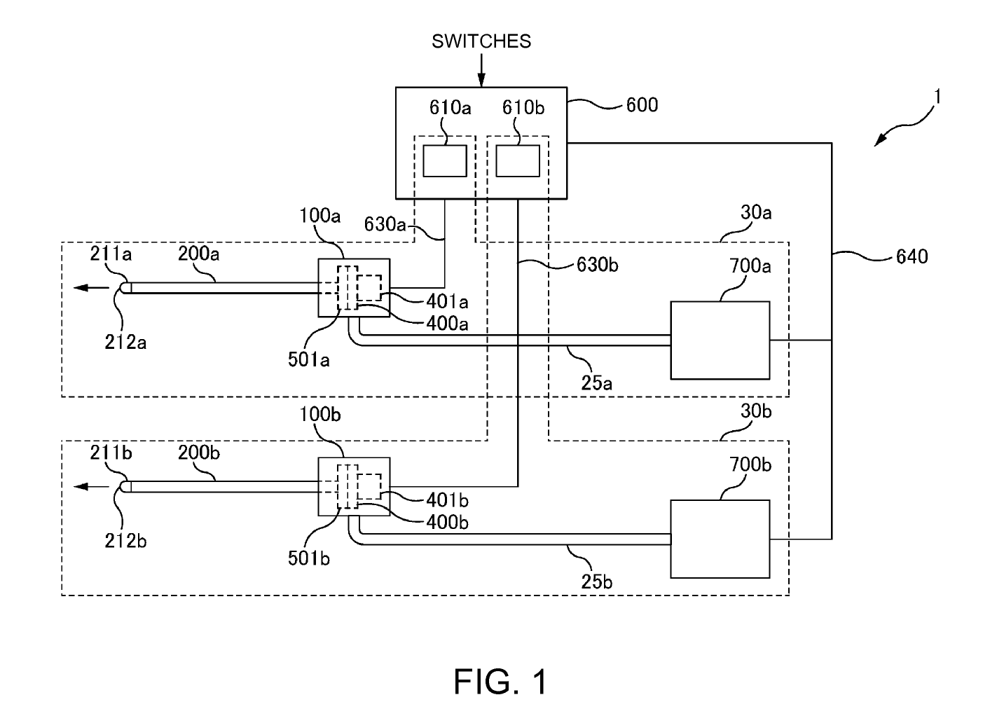

FIG. 1 is a schematic explanatory diagram showing a fluid ejection device 1 functioning as a surgical knife according to this embodiment. The fluid ejection device 1 according to this embodiment includes a driving control unit 600, a first pulsation generator (a first fluid ejection unit) 100a, a first pump (a first fluid supplying unit) 700a, a first connection tube (a first channel) 25a, a second pulsation generator (a second fluid ejection unit) 100b, a second pump (a second fluid supplying unit) 700b, and a second connection tube (a second channel) 25b.

The driving control unit 600 performs control of the fluid ejection device 1 in cooperation with the first pump 700a and the second pump 700b.

The first pump 700a supplies the fluid to the first pulsation generator 100a via the first connection tube 25a.

The second pump 700b supplies the fluid to the second pulsation generator 100b via the second connection tube 25b.

The first pulsation generator 100a ejects, in a pulse-like manner, the fluid supplied from the first pump 700a via the first connection tube 25a.

The second pulsation generator 100b ejects, in a pulse-like manner, the fluid supplied from the second pump 700b via the second connection tube 25b.

The driving control unit 600 and the first pulse generating unit 100a are connected by a first control cable 630a. A first driving-signal output unit 610a included in the driving control unit 600 transmits, to the first pulsation generator 100a via the first control cable 630a, a drive signal (a first drive signal) for ejecting the fluid from the first pulsation generator 100a in a pulse-like manner.

Similarly, the driving control unit 600 and the second pulsation generator 100b are connected by a second control able 630b. A second driving-signal output unit 610b included in the driving control unit 600 transmits, to the second pulsation generator 100b via the second control cable 630b, a drive signal (a second drive signal) for ejecting the fluid from the second pulsation generator 100b in a pulse-like manner.

The driving control unit 600, the first pump 700a, and the second pump 700b are connected by a communication cable 640. The driving control unit 600, the first pump 700a, and the second pump 700b exchange various commands and data one another according to a predetermined communication protocol such as a CAN (Controller Area Network).

As explained in detail below, among the components explained above, the first pulsation generator 100a, the first pump 700a, the first connection tube 25a, the first driving-signal output unit 610a, and the first control cable 630a are included in components 30a of a first group. The components 30a of the first group are components of the fluid ejection device 1 used for ejecting the fluid from the first pulsation generator 100a.

Similarly, the second pulsation generator 100b, the second pump 700b, the second connection tube 25b, the second driving-signal output unit 610b, and the second control cable 630b are included in components 30b of a second group. The components 30b of the second group are components of the fluid ejection device 1 used for ejecting the fluid from the second pulsation generator 100b.

The components 30b of the second group are auxiliary components used for ejecting the fluid instead of the components 30a of the first group when malfunction is detected in any one of the components 30a of the first group.

Note that, in the driving control unit 600, the first driving-signal output unit 610a used for ejecting the fluid from the first pulsation generator 100a is included in the components 30a of the first group and the second driving-signal output unit 610b used for ejecting the fluid from the second pulsation generator 100b is included in the components 30b of the second group.

Note that, in the following explanation, the functions and the structures of the first pulsation generator 100a and the second pulsation generator 100b are the same. Therefore, for simplification of explanation, the first pulsation generator 100a and the second pulsation generator 100b are collectively referred to as pulsation generators 100 as appropriate and explained except when it is particularly necessary to distinguish and explain the first pulsation generator 100a and the second pulsation generator 100b.

Similarly, the first pump 700a and the second pump 700b, the first connection tube 25a and the second connection tube 25b, and the first control cable 630a and the second control cable 630b are respectively collectively referred to as pumps 700, connection tubes 25, and control cables 630 as appropriate and explained except when it is particularly necessary to distinguish and explain the first pump 700a and the second pump 700b, the first connection tube 25a and the second connection tube 25b, and the first control cable 630a and the second control cable 630b.

On the other hand, when it is necessary to distinguish and explain the components 30a of the first group and the components 30b of the second group, for example, the components are distinguished and shown like the first pump 700a and the second pump 700b. Suffixes such as “a” and “b” are added to reference numerals. In this case, the suffixes “a” and “b” are also added to reference numerals of components in the first pump 700a. The suffixes “a” and “b” are also added to reference numerals of components of the second pump 700b.

As explained below in detail, the pulsation generators 100 include fluid chambers 501 in which the fluid supplied from the pumps 700 is stored, diaphragms 400 that change the volume of the fluid chambers 501, and piezoelectric elements 401 that vibrate the diaphragms 400.

The pulsation generators 100 include thin pipe-like fluid ejecting pipes 200 functioning as channels of the fluid ejected from the fluid chambers 501 and nozzles 211 with a reduced channel diameter attached to the distal end portions of the fluid ejecting pipes 200.

The pulsation generators 100 drive the piezoelectric elements 401 with a drive signal output from the driving control unit 600, change the volume of the fluid chambers 501 to apply pressure to the fluid in a pulse-like manner and convert the fluid into a pulsed flow, and eject the fluid in a pulse-like manner at high speed through the nozzles 211.

The driving control unit 600 receives inputs of signals from various switches operated by a surgeon or the like who performs a surgical operation using the pulsation generators 100. The driving control unit 600 controls the pumps 700 and the pulsation generators 100 via the control cables 630 and the communication cable 640.

As the switches connected to the driving control unit 600, there are, for example, a pulsation-generating-unit start switch 625, an ejecting-strength changeover switch 627, a flushing switch 628, and a pulsation-generating-unit changeover switch 629 (not shown in the figure).

The pulsation-generating-unit changeover switch 629 is a switch for selecting from which of the components 30a of the first group and the components 30b of the second group the fluid is ejected. The driving control unit 600 outputs a drive signal to the control cable 630 (the first control cable 630a or the second control cable 630b) connected to the selected pulsation generator 100 (the first pulsation generator 100a or the second pulsation generator 100b).

In this embodiment, when a surgical operation is performed using the fluid ejection device 1, first, the components 30a of the first group is used. Therefore, the pulsation-generating-unit changeover switch 629 is set by the operator to select the components 30a of the first group. When malfunction is detected in the components 30a of the first group while the surgical operation is performed using the components 30a of the first group, the fluid ejection device 1 switches the control to perform ejecting of the fluid using the components 30b of the second group.

The pulsation-generating-unit start switch 625 is a switch for switching presence or absence of ejecting of the fluid from the pulsation generators 100. When the pulsation-generating-unit start switch 625 is operated by the surgeon who performs a surgical operation using the pulsation generators 100, the driving control unit 600 executes, in cooperation with the pump 700, control for ejecting the fluid or stopping the ejecting of the fluid from the pulsation generator 100 selected by the pulsation-generating-unit changeover switch 629. The pulsation-generating-unit start switch 625 can take a form of a footswitch operated by the foot of the surgeon or can take a form of being disposed integrally with the pulsation generators 100, which are gripped by the surgeon, and operated by the hand and the fingers of the surgeon.

The ejecting-strength changeover switch 627 is a switch for changing ejecting strength of the fluid ejected from the pulsation generators 100. When the ejecting-strength-changeover switch 627 is operated, the driving control unit 600 applies control for increasing or reducing the ejecting strength of the fluid to the pulsation generators 100 and the pumps 700.

The flushing switch 628 is explained below.

In this embodiment, the pulsed flow means flowing of the fluid that flows in a fixed direction and involves cyclic or irregular fluctuation of a flow rate or flow velocity of the fluid. The pulsed flow also includes an intermittent flow that repeats flowing and stop of the fluid. However, since the flow rate or the flow velocity of the fluid only has to cyclically or irregularly fluctuates, the pulsed flow does not always need to be the intermittent flow.

Similarly, ejecting the fluid in a pulse-like manner means ejecting of the fluid, the flow rate or the flow velocity of which cyclically or irregularly fluctuates. Examples of the pulse-like ejecting include intermittent ejecting that repeats ejecting and non-ejecting of the fluid. However, since the flow rate or the flow velocity of the ejected fluid only has to cyclically or irregularly fluctuates, the ejecting of the fluid does not always need to be intermittent ejecting.

When the pulsation generators 100 stop the driving, that is, when the pulsation generators 100 do not change the volume of the fluid chambers 501, the fluid supplied from the pumps 700 functioning as fluid supplying units at predetermined pressure continuously flows out from the nozzles 211 through the fluid chambers 501.

### Pump

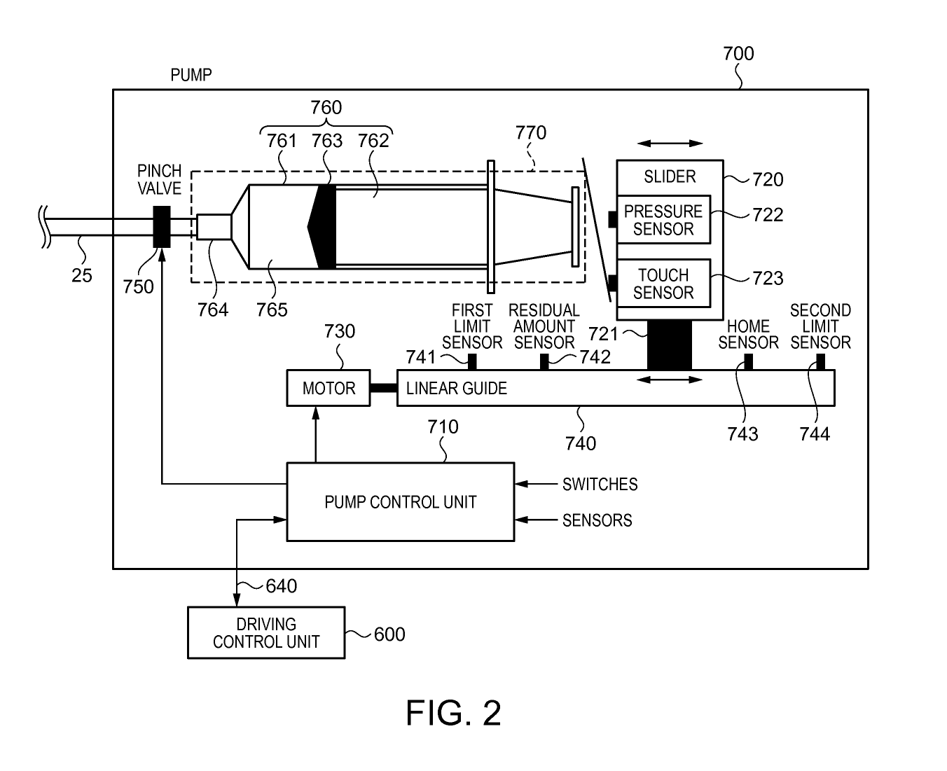

An overview of the configuration and the operation of the pump 700 according to this embodiment is explained with reference to FIG. 2.

As explained above, the fluid ejection device 1 according to this embodiment includes the first pump 700a and the second pump 700b. The first pump 700a and the second pump 700b are collectively referred to as pump 700 and explained.

The pump 700 according to this embodiment includes a pump control unit 710, a slider 720, a motor 730, a linear guide 740, and a pinch valve 750. The pump 700 includes a fluid-container attaching unit 770 for detachably attaching a fluid container 760 that stores the fluid. The fluid-container attaching unit 770 is formed to hold the fluid container 760 in a specified position when the fluid container 760 is attached.

As explained in detail below, signals from a slider release switch 780, a slider set switch 781, a fluid-feed ready switch 782, a priming switch 783, and a pinch valve switch 785 are connected to the pump control unit 710 (not shown in the figure).

In this embodiment, as an example, the fluid container 760 is configured as an injection cylinder including a syringe 761 and a plunger 762.

In the fluid container 760, an opening section 764 having a projected cylindrical shape is formed at the distal end portion of the syringe 761. When the fluid container 760 is attached to the fluid-container attaching unit 770, an end portion of the connection tube 25 is fit in the opening section 764 to forma channel of the fluid from the inside of the syringe 761 to the connection tube 25.

The pinch valve 750 is a valve that is provided on a route of the connection tube 25 and opens and closes a channel of the fluid between the fluid container 760 and the pulsation generator 100.

Opening and closing of the pinch valve 750 is performed by the control unit 710. When the pump control unit 710 opens the pinch valve 750, the fluid container 760 and the pulsation generator 100 communicate with each other through the channel. When the pump control unit 710 closes the pinch valve 750, the channel between the fluid container 760 and the pulsation generator 100 is blocked.

After the fluid container 760 is attached to the fluid-container attaching unit 770, when the plunger 762 of the fluid container 760 is moved in a direction for pushing the plunger 762 into the syringe 761 (hereinafter also referred to as push-in direction) in a state in which the pinch valve 750 is opened, the volume of a space (hereinafter also referred to as fluid storing section 765) surrounded by an end face of a gasket 763, which is made of resin such as rubber having elasticity, attached to the distal end on the push-in direction side of the plunger 762 and the inner wall of the syringe 761 decreases. The fluid filled in the fluid storing section 765 is ejected from the opening section 764 at the distal end portion of the syringe 761. The fluid ejected from the opening section 764 is filled in the connection tube 25 and supplied to the pulsation generator 100.

On the other hand, after the fluid container 760 is attached to the fluid-container attaching unit 770, when the plunger 762 of the fluid container 760 is moved in the push-in direction in a state in which the pinch valve 750 is closed, the volume of the fluid storing unit 765 surrounded by the gasket 763 attached to the distal end of the plunger 762 and the inner wall of the syringe 761 decreases. The pressure of the fluid filled in the fluid storing unit 765 can be increased.

The movement of the plunger 762 is performed by the pump control unit 710 moving the slider 720 along a direction in which the plunger 762 slides when the fluid container 760 is attached to the fluid-container attaching unit 770 (the push-in direction and the opposite direction of the push-in direction).

Specifically, the slider 720 is attached to the linear guide 740 to engage a pedestal section 721 of the slider 720 to a rail (not shown in the figure) linearly formed in the linear guide 740 along the sliding direction of the plunger 762. The linear guide 740 moves the pedestal section 721 of the slider 720 along the rail using power transmitted from the motor 730 driven by the pump control unit 710, whereby the slider 720 moves in the sliding direction of the plunger 762.

As shown in FIG. 2, a first limit sensor 741, a residual amount sensor 742, a home sensor 743, and a second limit sensor 744 are provided along the rail of the linear guide 740.

All of the first limit sensor 741, the residual amount sensor 742, the home sensor 743, and the second limit sensor 744 are sensors that detect the position of the slider 720 that moves on the rail of the linear guide 740. Signals detected by the sensors are input to the pump control unit 710.

The home sensor 743 is a sensor used for determining an initial position (hereinafter also referred to as home position) of the slider 720 on the linear guide 740. The home position is a position where the slider 720 is held when work such as attachment and replacement of the fluid container 760 is performed.

The residual amount sensor 742 is a sensor for detecting the position of the slider 720 (hereinafter also referred to as residual amount position) where the residual amount of the fluid in the fluid container 760 is equal to or smaller than a predetermined value when the slider 720 moves in the push-in direction of the plunger 762 from the home position. When the slider 720 moves to the residual amount position where the residual amount sensor 742 is provided, predetermined an alarm is output to the operator (the surgeon or an assistant). The operator determines to perform work for replacing the fluid container 760 currently in use with a new fluid container 760 at appropriate timing. Alternatively, when the auxiliary second pump 700b having the same configuration as the pump 700 (the first pump 700a) is prepared, switching work is performed to supply the fluid to the pulsation generator 100 from the auxiliary second pump 700b.

The first limit sensor 741 indicates a limit position (hereinafter also referred to as first limit position) in a movable range of the slider 720 moving in the push-in direction of the plunger 762 from the home position. When the slider 720 moves to the first limit position where the first limit sensor 741 is provided, the residual amount of the fluid in the fluid container 760 is smaller than the residual amount at the time when the slider 720 is in the residual amount position. A predetermined alarm is output to the operator. In this case as well, the work for replacing the fluid container 760 currently in use with the new fluid container 760 or the switching work to the auxiliary second pump 700b is performed.

On the other hand, the second limit sensor 744 indicates a limit position (hereinafter also referred to as second limit position) of the movable range of the slider 720 moving in the opposite direction of the push-in direction of the plunger 762 from the home position. The predetermined alarm is also output when the slider 720 moves to the second limit position where the second limit sensor 744 is provided.

A touch sensor 723 and a pressure sensor (a pressure detecting unit) 722 are attached to the slider 720.

The touch sensor 723 is a sensor for detecting whether the slider 720 is in contact with the plunger 762 of the fluid container 760.

The pressure sensor 722 is a sensor that detects the pressure of the fluid in the fluid storing unit 765 formed by the inner wall of the syringe 761 and the gasket 763, that is, the pressure of the slider 720 in pressing the fluid storing unit 765 and outputs a signal (a detection signal) of a level (e.g., a voltage, an electric current, or a frequency) corresponding to the pressure.

When the slider 720 is moved in the push-in direction in a state in which the pinch valve 750 is closed, after the slider 720 comes into contact with the plunger 762, the pressure of the fluid in the fluid storing unit 765 rises as a push-in amount of the slider 720 is increased.

On the other hand, when the slider 720 is moved in the push-in direction in a state in which the pinch valve 750 is opened, even after the slider 720 comes into contact with the plunger 762, the fluid in the fluid storing unit 765 flows out from the nozzle 211 of the pulsation generator 100 through the connection tube 25. Therefore, the pressure of the fluid in the fluid storing unit 765 rises to a certain degree but does not rise even if the slider 720 is further moved in the push-in direction.

Note that signals from the touch sensor 723 and the pressure sensor 722 are input to the pump control unit 710.

In the following explanation, the slider 720, the motor 730, and the linear guide 740 are sometimes referred to as fluid pressing unit 731. The fluid pressing unit 731 presses the fluid storing unit 765 and causes the fluid to flow out from the opening section 764 of the fluid container 760.

A preparation operation (a first preparation operation and a second preparation operation) for attaching the fluid container 760, in which the fluid is filled, to the fluid-container attaching unit 770 anew, supplying the fluid in the fluid container 760 to the pulsation generator 100, and enabling the fluid to be ejected from the pulsation generator 100 in a pulse-like manner is explained.

The first preparation operation is a preparation operation performed for enabling ejecting of the fluid from the components 30a of the first group. The second preparation operation is a preparation operation performed for enabling ejecting of the fluid from the components 30b of the second group.

The preparation operation is an operation for changing the fluid in the channel to a predetermined state (explained below) to eject the fluid from the pulsation generator 100 at appropriate strength.

The pump control unit 710 and the driving control unit 600 execute the preparation operation according to an instruction input (explained below) to the fluid ejection device 1 by the operator. However, for example, when malfunction is detected in the components 30a of the first group, the pump control unit 710 and the driving control unit 600 can execute the preparation operation according to a state of the fluid ejection device 1 even if there is no instruction input.

When performing the preparation operation according to the instruction input by the operator, the pump control unit 710 and the driving control unit 600 performs the preparation operation for any one of the components 30a of the first group and the components 30b of the second group designated by the pulsation-generating-unit changeover switch 629.

In this embodiment, first, the ejecting of the fluid is performed using the components 30a of the first group. The components 30b of the second group are used as backup in case where malfunction occurs in the components 30a of the first group. Therefore, usually, the pulsation-generating-unit changeover switch 629 is set to select the components 30a of the first group.

The preparation operation includes various kinds of processing such as preliminary pressurization, priming processing, and flushing processing. The pump control unit 710 and the driving control unit 600 can execute the preparation operation in various combinations of these kinds of processing according to a state of the fluid ejection device 1.

For example, as the preparation operation, only the preliminary pressurization is performed, only the priming processing is performed, the preliminary pressurization and the priming processing are performed, or the preliminary pressurization, the priming processing, and the flushing processing are performed.

In the following explanation, as an example, according to an instruction input from the operator, for the components 30a of the first group, the pump control unit 710 performs the preliminary pressurization and then performs the priming processing. Thereafter, the pump control unit 710 and the driving control unit 600 perform the flushing processing in cooperation with each other.

Note that the preparation operation explained below is performed in the same manner for the components 30a of the first group and the components 30b of the second group. Therefore, the preparation operation performed for the components 30a of the first group and the preparation operation performed for the components 30b of the second group are collectively explained.

First, the operator operates the slider release switch 780 to input an ON signal of the slider release switch 780 to the pump control unit 710. Then, the pump control unit 710 moves the slider 720 to the home position.

The operator attaches the fluid container 760, which is connected to the connection tube 25 beforehand, to the fluid-container attaching unit 770. Note that the fluid is already filled in the syringe 761 of the fluid container 760.

After setting the connection tube 25 in the pinch valve 750, when the operator operates the pinch valve switch 785 to input an ON signal of the pinch valve switch 785 to the pump control unit 710, the pump control unit 710 closes the pinch valve 750.

Subsequently, the operator operates the slider set switch 781 to input an ON signal of the slider set switch 781 to the pump control unit 710. Then, the pump control unit 710 moves the slider 720 in the push-in direction and starts control such that the pressure of the fluid stored in the fluid storing unit 765 in the fluid container 760 is within a specified range (hereinafter also referred to as rough window) with respect to a predetermined target pressure value.

As explained above, the preliminary pressurization is processing for setting the pressure of the fluid stored in the fluid storing unit 765 within the specified range (i.e., equal to or higher than a predetermined value (a first predetermined value and a second predetermined value)).

Thereafter, when the fluid-feed ready switch 782 is pressed by the operator, an ON signal of the fluid-feed ready switch 782 is input to the pump control unit 710. When the pressure of the fluid in the fluid storing unit 765 is in the rough window, the pump control unit 710 changes to a fluid feedable state in which feeding of the fluid from the pump 700 to the pulsation generator 100 is permitted.

In the fluid feedable state of the pump control unit 710, when an ON signal of the priming switch 783 is input to the pump control unit 710 by the operation by the operator, the pump control unit 710 starts the priming processing. The priming processing is processing for causing the fluid in the fluid container 760 to reach a fluid-ejecting opening section 212 of the pulsation generator 100 via the connection tube 25 and fill a channel from the fluid container 760 to the fluid-ejecting opening section 212 with the fluid.

When the priming processing is started, the pump control unit 710 opens the pinch valve 750 and starts movement in the push-in direction of the slider 720 at timing simultaneous or substantially simultaneous with the opening of the pinch valve 750 (e.g., with a time difference of about several milliseconds to several tens milliseconds). The movement of the slider 720 is performed at predetermined speed at which a delivery amount per unit time of the fluid from the fluid container 760 is fixed. The priming processing is performed until a predetermined time equal to or longer than time required by the fluid in the fluid container 760 to reach the fluid-ejecting opening section 212 of the pulsation generator 100 elapses (or the slider 720 moves a predetermined distance sufficient for the fluid in the fluid container 760 to reach the fluid-ejecting opening section 212 of the pulsation generator 100) or until the operator operates the priming switch 783 to input an OFF signal.

Consequently, a predetermined amount of the fluid in the fluid storing unit 765 is delivered from the pump 700 at predetermined flow velocity (an ejection amount of the fluid per unit time) and fills the inside of the connection tube 25 from the pinch valve 750 to the pulsation generator 100 and also fills the fluid chamber 501 of the pulsation generator 100, the fluid ejecting pipe 200, and the like. Note that the air present in the connection tube 25 and the pulsation generator 100 before the start of the priming processing is emitted to the atmosphere from the nozzle 211 of the pulsation generator 100 as the fluid flows into the connection tube 25 and the pulsation generator 100.

Note that the predetermined speed, the predetermined distance, or the predetermined time for moving the slider 720 in the priming processing is stored in the pump control unit 710 beforehand.

In this way, the priming processing is completed.

Subsequently, when an ON signal of the flushing switch 628 is input to the driving control unit 600 by the operation by the operator, the driving control unit 600 and the pump control unit 710 start degassing processing.

The degassing processing is processing for discharging air bubbles remaining in the connection tube 25 and the pulsation generator 100 from the nozzle 211 of the pulsation generator 100 and removing the air bubbles from the channel.

In the degassing processing, in a state in which the pinch valve 750 is opened, the pump control unit 710 moves the slider 720 in the push-in direction at predetermined speed for fixing a delivery amount per unit time of the fluid from the fluid container 760, that is, speed for setting a flow rate of the fluid flowing in a predetermined time in the channel to a predetermined amount and supplies the fluid to the pulsation generator 100. The driving control unit 600 drives the piezoelectric element 401 of the pulsation generator 100 in association with the ejection of the fluid by the pump 700 and ejects the fluid from the pulsation generator 100 in a pulse-like manner. Consequently, the air bubbles remaining in the connection tube 25 and the pulsation generator 100 are discharged from the nozzle 211 of the pulsation generator 100. The degassing processing is performed until a predetermined time elapses (or the slider 720 moves a predetermined distance) or until the operator operates the flushing switch 628 to input an OFF signal.

Note that the predetermined speed, the predetermined time, or the predetermined distance for moving the slider 720 in the degassing processing is stored in the driving control unit 600 and the pump control unit 710 beforehand.

In this way, the preparation operation (the preliminary pressurization, the priming processing, and the degassing processing) is completed.

When the preparation operation ends, the pump control unit 710 closes the pinch valve 750 and detects the pressure of the fluid stored in the fluid storing unit 765 of the fluid container 760. The pump control unit 710 performs control for adjusting the position of the slider 720 such that the pressure falls within the rough window.

Thereafter, if the pressure of the fluid in the fluid storing unit 765 is within the rough window, the fluid can be ejected from the pulsation generator 100 in a pulse-like manner.

In this state, when the pulsation-generating-unit start switch 625 is operated by the foot of the surgeon and an ON signal of the pulsation-generating-unit start switch 625 is input to the driving control unit 600, according to a signal transmitted from the driving control unit 600, the pump control unit 710 opens the pinch valve 750, moves the slider 720 in the push-in direction at predetermined speed at timing simultaneous or substantially simultaneous with the opening of the pinch valve 750 (e.g., with a time difference of several milliseconds to several tens milliseconds), and starts the supply of the fluid to the pulsation generator 100. On the other hand, the driving control unit 600 starts the driving of the piezoelectric element 401 and changes the volume of the fluid chamber 501 to generate a pulsed flow. In this way, the fluid is ejected from the nozzle 211 at the distal end of the pulsation generator 100 in a pulse-like manner at high speed.

Thereafter, when the surgeon operates the pulsation-generating-unit start switch 625 by foot and an OFF signal of the pulsation-generating-unit start switch 625 is input to the driving control unit 600, the driving control unit 600 stops the driving of the piezoelectric element 401. According to a signal transmitted from the driving control unit 600, the pump control unit 710 stops the movement of the slider 720 and closes the pinch valve 750. In this way, the ejecting of the fluid from the pulsation generator 100 stops.

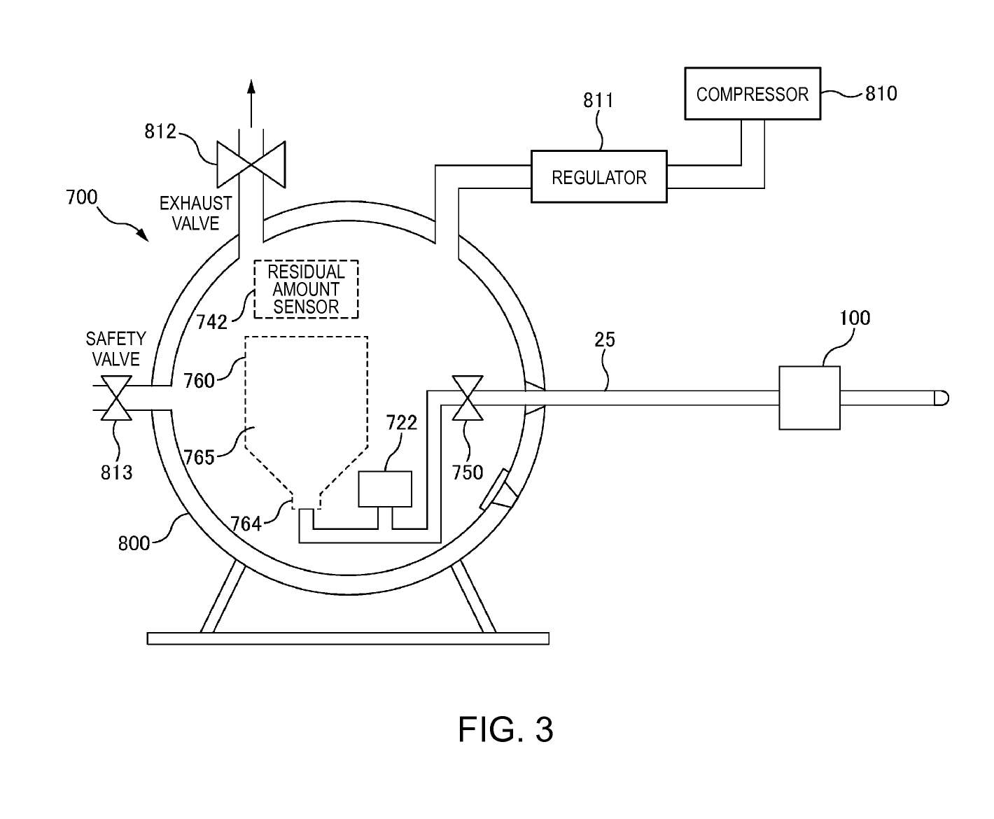

Note that, the pump 700 according to this embodiment has the configuration in which the slider 720 presses the fluid container 760 configured as the injection cylinder including the syringe 761 and the plunger 762. However, the pump 700 may have a configuration shown in FIG. 3.

The pump 700 shown in FIG. 3 has a configuration in which the fluid container 760 configured as an infusion fluid bag, which stores the fluid, is attached in a pressurization chamber 800 and, after the air supplied from a compressor 810 is smoothed by a regulator 811, the air is pressure-fed into the pressurization chamber 800 to press the fluid container 760.

In a state in which the air in the pressurization chamber 800 is pressurized to press the fluid container 760, when the pinch valve 750 is opened, the fluid stored in the fluid storing unit 765 of the fluid container 760 flows out from the opening section 764 and is supplied to the pulsation generator 100 through the connection tube 25.

Note that the air in the pressurization chamber 800 is emitted to the atmosphere by opening an exhaust valve 812. When the pressure of the air in the pressurization chamber 800 exceeds predetermined pressure, even if the exhaust valve 812 is not opened, the air in the pressurization chamber 800 is emitted to the atmosphere when a safety valve 813 opens.

Note that, although not shown in FIG. 3, the compressor 810, the regulator 811, the exhaust valve 812, and the pinch valve 750 are controlled by the pump control unit 710.

Detection signals output from the pressure sensor 722 that detects the pressure of the fluid in the fluid container 760 and the residual amount sensor 742 that detects the residual amount of the fluid in the fluid container 760 are also input to the pump control unit 710.

In the case of the pump 700 shown in FIG. 3, the compressor 810, the regulator 811, and the pressurization chamber 800 configure the fluid pressing unit 731.

By adopting the pump 700 having such a form, it is possible to increase an amount of the fluid that can be supplied to the pulsation generator 100 per unit time. It is also possible to supply the fluid at high pressure with the pulsation generator 100. Further, since the infusion fluid bag, which stores the fluid, is directly used as the fluid container 760, it is possible to prevent contamination of the fluid. It is also possible to continuously feed the fluid to the pulsation generator 100 without causing pulsation.

Besides, in this embodiment, the driving control unit 600 is disposed in a position separated from the pump 700 and the pulsation generator 100. However, the driving control unit 600 may be configured integrally with the pump 700.

When a surgical operation is performed using the fluid ejection device 1, a part griped by the surgeon is the pulsation generator 100. Therefore, the connection tube 25 to the pulsation generator 100 is desirably as flexible as possible. It is desirable that the connection tube 25 is a flexible and thin tube and the ejection pressure of the fluid from the pump 700 is set to low pressure in a range in which the fluid can be fed to the pulsation generator 100. Therefore, the ejection pressure of the pump 700 is set to approximately 0.3 atm (0.03 MPa) or less.

In particular, when there is a risk that a failure of an apparatus causes a serious accident as in brain surgery, spouting of high-pressure fluid in cutting or the like of the connection tube 25 has to be avoided. Therefore, it is also requested to keep the ejection pressure from the pump 700 at low pressure.

### Pulsation Generator

The structure of the pulsation generator 100 according to this embodiment is explained.

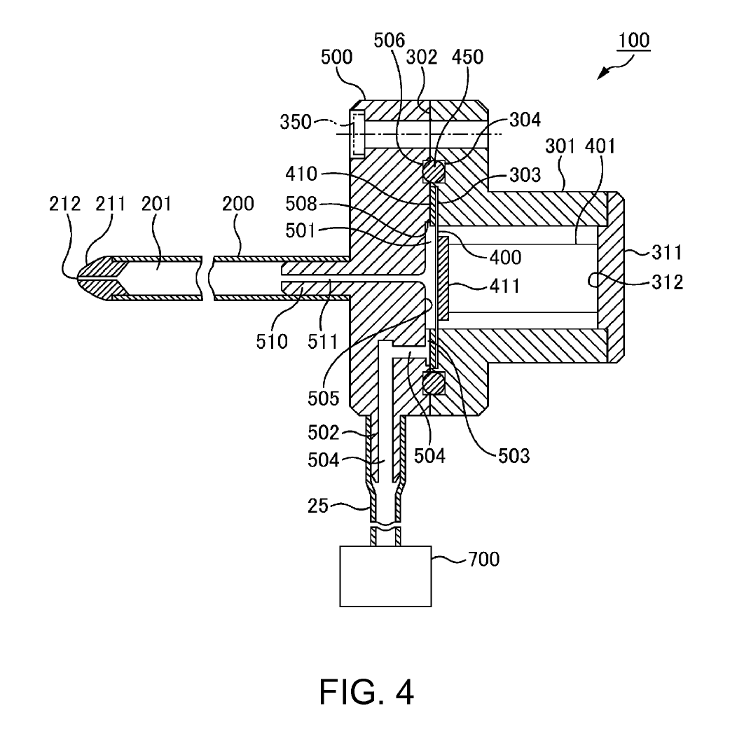

FIG. 4 is a sectional view showing the structure of the pulsation generator 100 according to this embodiment. In FIG. 4, the fluid ejecting pipe 200 including a pulsation generating unit configured to generate pulsation of the fluid and including a connection channel 201 functioning as a channel for ejecting the fluid is connected to the pulsation generator 100.

In the pulsation generator 100, an upper case 500 and a lower case 301 are respectively joined on surfaces opposed to each other. The upper case 500 and the lower case 301 are screwed by four fixing screws 350 (not shown in the figure). The lower case 301 is a cylindrical member having a brim section. One end portion of the lower case 301 is closed by a bottom plate 311. The piezoelectric element 401 is disposed in the inner space of the lower case 301.

The piezoelectric element 401 is a stacked piezoelectric element and configures an actuator. One end portion of the piezoelectric element 401 is fixedly attached to the diaphragm 400 via a top plate 411. The other end portion of the piezoelectric element 401 is fixedly attached to an upper surface 312 of the bottom plate 311.

The diaphragm 400 is made of a disk-like metal thin plate. In a recessed section 303 of the lower case 301, a circumferential edge portion of the diaphragm 400 is closely attached and fixedly attached to the bottom surface of a recessed section 303. By inputting a drive signal to the piezoelectric element 401 functioning as a volume varying unit, the volume of the fluid chamber 501 is changed via the diaphragm 400 according to expansion and contraction of the piezoelectric element 401.

On the upper surface of the diaphragm 400, a reinforcing plate 410 made of a disk-like metal thin plate having an opening section in the center is stacked and disposed.

In the upper case 500, a recessed section is formed in the center of the surface opposed to the lower case 301. A rotating body shape configured from the recessed section and the diaphragm 400 and filled with the fluid is the fluid chamber 501. That is, the fluid chamber 501 is a space surrounded by a sealing surface 505 and an inner circumferential sidewall 508 of the recessed section of the upper case 500 and the diaphragm 400. An outlet channel 511 is drilled in substantially the center of the fluid chamber 501.

The outlet channel 511 is pierced from the fluid chamber 501 to an end portion of an outlet cannel pipe 510 projected from one end face of the upper case 500. A connecting section of the outlet channel 511 to the sealing surface 505 of the fluid chamber 501 is smoothly rounded in order to reduce fluid resistance.

Note that, in this embodiment (see FIG. 4), the shape of the fluid chamber 501 explained above is a substantially cylindrical shape sealed at both ends. However, the shape may be a conical shape or a trapezoidal shape or may be a semispherical shape or the like in side view and is not limited to the cylindrical shape. For example, if the connecting section of the outlet channel 511 and the sealing surface 505 is formed in a shape like a funnel, it is easy to discharge air bubbles in the fluid chamber 501 explained below.

The fluid ejecting pipe 200 is connected to the outlet channel pipe 510. The connection channel 201 is drilled in the fluid ejecting pipe 200. The diameter of the connection channel 201 is larger than the diameter of the outlet channel 511. The thickness of a pipe section of the fluid ejecting pipe 200 is set in a range in which the pipe section has rigidity for not absorbing pressure pulsation of the fluid.

The nozzle 211 is inserted into the distal end portion of the fluid ejecting pipe 200. The fluid-ejecting opening section 212 is drilled in the nozzle 211. The diameter of the fluid-ejecting opening section 212 is smaller than the diameter of the connection channel 201.

On the side surface of the upper case 500, an inlet channel pipe (a fluid intake port) 502, into which the connection tube 25 for supplying the fluid from the pump 700 is inserted, is projected. A connection channel 504 on an inlet channel side is drilled in the inlet channel pipe 502. The connection channel 504 communicates with an inlet channel 503. The inlet channel 503 is formed in a groove shape in the circumferential edge portion of the sealing surface 505 of the fluid chamber 501 and communicates with the fluid chamber 501.

On the joining surface of the upper case 500 and the lower case 301, in a separated position in the outer circumferential direction of the diaphragm 400, a packing box 304 is formed on the lower case 301 side and a packing box 506 is formed on the upper case 500 side. A ring-like packing 450 is attached in a space formed by the packing boxes 304 and 506.

When the upper case 500 and the lower case 301 are assembled, the circumferential edge portion of the diaphragm 400 and the circumferential edge portion of the reinforcing plate 410 are closely set in contact with the circumferential edge portion of the sealing surface 505 of the upper case 500 by the bottom surface of the recessed section 303 of the lower case 301. In this case, the packing 450 is pressed by the upper case 500 and the lower case 301 to prevent a fluid leak from the fluid chamber 501.

When the fluid is ejected, the inside of the fluid chamber 501 is in a high pressure state of 30 atm (3 MPa) or higher. It is likely that the fluid slightly leaks in joining sections of the diaphragm 400, the reinforcing plate 410, the upper case 500, and the lower case 301. However, the leak is prevented by the packing 450.

When the packing 450 is disposed as shown in FIG. 4, the packing 450 is compressed by the pressure of the fluid leaking from the fluid chamber 501 at high pressure. The packing 450 is more strongly pressed against the walls in the packing boxes 304 and 506. Therefore, it is possible to more surely prevent the leak of the fluid. Consequently, it is possible to maintain a high pressure rise in the fluid chamber 501 during driving.

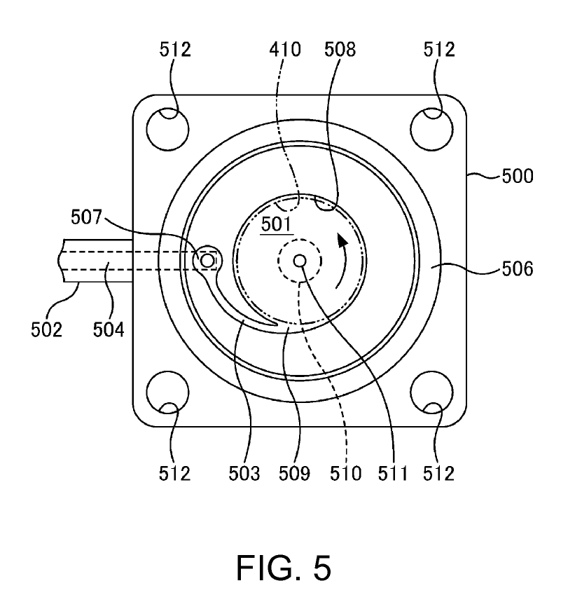

The inlet channel 503 formed in the upper case 500 is explained more in detail with reference to FIG. 5.

FIG. 5 is a plan view showing a form of the inlet channel 503. A state in which the upper case 500 is viewed from the joining surface side with the lower case 301 is shown.

In FIG. 5, the inlet channel 503 is formed in a circumferential edge groove shape of the sealing surface 505 of the upper case 500.

One end portion of the inlet channel 503 communicates with the fluid chamber 501. The other end portion of the inlet channel 503 communicates with the connection channel 504. A fluid reservoir 507 is formed in a connecting section of the inlet channel 503 and the connection channel 504. A connecting section of the fluid reservoir 507 and the inlet channel 503 is smoothly rounded to reduce fluid resistance.

The inlet channel 503 communicates with the inner circumferential sidewall 508 of the fluid chamber 501 toward a substantially tangential direction. The fluid supplied from the pump 700 (see FIG. 1) at predetermined pressure flows along the inner circumferential sidewall 508 (in a direction indicated by an arrow in FIG. 5) to generate a swirl flow in the fluid chamber 501. The swirl flow is pressed to the inner circumferential sidewall 508 side with a centrifugal force by swirling. The air bubbles included in the fluid chamber 501 concentrate on the center of the swirl flow.

The air bubbles collected in the center are removed from the outlet channel 511. Therefore, it is more desirable to provide the outlet channel 511 in the vicinity of the center of the swirl flow, that is, in the axial center of a rotating body shape.

As shown in FIG. 5, the inlet channel 503 is curved. The inlet channel 503 may communicate with the fluid chamber 501 along a straight line without being curved. However, the inlet channel 503 is curved to increase channel length and obtain desired inertance (explained below) in a narrow space.

Note that, as shown in FIG. 5, the reinforcing plate 410 is disposed between the diaphragm 400 and the circumferential edge portion of the sealing surface 505 in which the inlet channel 503 is formed. The reinforcing plate 410 is provided to improve durability of the diaphragm 400. A cutout-like connection opening section 509 is formed in a connecting section of the inlet channel 503 to the fluid chamber 501. Therefore, when the diaphragm 400 is driven at a high frequency, it is likely that stress concentration occurs in the vicinity of the connection opening section 509 to cause fatigue fracture. Therefore, the reinforcing plate 410 having a continuous opening section without a cutout section is disposed to prevent stress concentration from occurring in the diaphragm 400.

In the outer circumferential corner portions of the upper case 500, screw holes 512 are opened in four places. The upper case 500 and the lower case 301 are screwed and joined in the positions of the screw holes.

Note that, although not shown in the figure, the reinforcing plate 410 and the diaphragm 400 can be joined and integrally stacked and fixedly attached. A method of fixedly attaching the reinforcing plate 410 and the diaphragm 400 may be a method of sticking the reinforcing plate 410 and the diaphragm 400 using an adhesive or may be a method such as solid phase diffusion joining or welding. However, the reinforcing plate 410 and the diaphragm 400 are more desirably closely attached on a joining surface.

### Operation of the Pulsation Generator

The operation of the pulsation generator 100 in this embodiment is explained with reference to FIGS. 1 to 5. Fluid ejection by the pulsation generator 100 in this embodiment is performed by a difference between inertance L1 (sometimes referred to as combined inertance L1) on the inlet channel 503 side and inertance L2 (sometimes referred to as combined inertance L2) on the outlet channel 511 side.

### Inertance

First, the inertance is explained.

When the density of the fluid is represented as ρ, the sectional area of a channel is represented as S, and the length of the channel is represented as h, inertance L is represented by L=ρ×h/S. When a pressure difference of the channel is represented as ΔP and a flow rate of the fluid flowing through the channel is represented as Q, by transforming an equation of motion in the channel using the inertance L, a relation of ΔP=L×dQ/dt is derived.

That is, the inertance L indicates a degree of influence on a temporal change of the flow rate. The temporal change of the flow rate is smaller as the inertance L is larger. The temporal change of the flow rate is larger as the inertance L is smaller.

Combined inertance concerning parallel connection of a plurality of channels and series connection of a plurality of channels having different shapes can be calculated by combining inertances of respective channels in the same manner as parallel connection or series connection of inductances in an electric circuit.

Note that, since the diameter of the connection channel 504 is set sufficiently large with respect to the diameter of the inlet channel 503, the inertance L1 on the inlet channel 503 side is calculated in a range of the inlet channel 503. In this case, since the connection tube 25 that connects the pump 700 and the inlet channel 503 has flexibility, the connection tube 25 may be excluded from the calculation of the inertance L1.

Since the diameter of the connection channel 201 is far larger than the diameter of the outlet channel 511 and the thickness of the pipe section (the pipe wall) of the fluid ejecting pipe 200 is small, the influence of the diameter of the connection channel 201 and the thickness of the pipe section of the fluid ejecting pipe 200 on the inertance L2 is very small. Therefore, the inertance L2 on the outlet channel 511 side may be replaced with the inertance of the outlet channel 511.

Note that the pipe wall of the fluid ejecting pipe 200 has sufficient rigidity for pressure propagation of the fluid.

In this embodiment, the channel length and the sectional area of the inlet channel 503 and the channel length and the sectional area of the outlet channel 511 are set such that the inertance L1 on the inlet channel 503 side is larger than the inertance L2 on the outlet channel 511 side.

### Ejecting of the Fluid

The operation of the pulsation generator 100 is explained below.

The fluid is supplied to the inlet channel 503 by the pump 700 at given pressure. As a result, when the piezoelectric element 401 does not perform an operation, the fluid flows in the fluid channel 501 with a difference between an ejection force of the pump 700 and a fluid resistance value of the entire inlet channel 503 side.

When a drive signal is input to the piezoelectric element 401 and the piezoelectric element 401 suddenly expands, the pressure in the fluid chamber 501 quickly rises and reaches several tens atm if the inertances L1 and L2 on the inlet channel 503 side and the outlet channel 511 side have sufficient magnitude.

The pressure in the fluid chamber 501 is far larger than the pressure by the pump 700 applied to the inlet channel 503. Therefore, inflow of the fluid into the fluid chamber 501 from the inlet channel 503 side decreases and outflow from the outlet channel 511 increases because of the pressure.

Since the inertance L1 of the inlet channel 503 is larger than the inertance L2 of the outlet channel 511, an increase amount of the fluid ejected from the outlet channel 511 is larger than a decrease amount of the flow rate of the fluid flowing into the fluid chamber 501 from the inlet channel 503. Therefore, pulse-like fluid ejection, that is, a pulsed flow occurs in the connection channel 201. Pressure fluctuation in the ejection propagates through the fluid ejecting pipe 200. The fluid is ejected from the fluid-ejecting opening section 212 of the nozzle 211 at the distal end.

Since the diameter of the fluid-ejecting opening section 212 of the nozzle 211 is smaller than the diameter of the outlet channel 511, the fluid is ejected as pulse-like droplets at higher pressure and higher speed.

On the other hand, the inside of the fluid chamber 501 changes to a decompressed state immediately after a pressure rise because of interaction of a decrease in a fluid inflow amount from the inlet channel 503 and an increase in a fluid outflow from the outlet channel 511. As a result, a flow of the fluid in the inlet channel 503 flowing to the fluid chamber 501 at speed same as the speed before the operation of the piezoelectric element 401 is restored after the elapse of a predetermined time by both of the pressure of the pump 700 and the decompressed state in the fluid chamber 501.

After the flow of the fluid in the inlet channel 503 is restored, if the piezoelectric element 401 expands, it is possible to continuously eject the pulsed flow from the nozzle 211.

### Removal of the Air Bubbles

A removing operation for the air bubbles in the fluid chamber 501 is explained.

As explained above, the inlet channel 503 communicates with the fluid chamber 501 through the route approaching the fluid chamber 501 while turning around the fluid chamber 501. The outlet channel 511 is opened in the vicinity of the rotation axis of the substantial rotating body shape of the fluid chamber 501.

Therefore, the fluid flowing into the fluid chamber 501 from the inlet channel 503 swirls along the inner circumferential sidewall 508 in the fluid chamber 501. The fluid is pressed to the inner circumferential sidewall 508 side of the fluid chamber 501 by a centrifugal force. Air bubbles included in the fluid concentrate on the center of the fluid chamber 501. As a result, the air bubbles are discharged from the outlet channel 511.

Therefore, even in a very small volume change of the fluid chamber 501 due to the piezoelectric element 401, the pressure fluctuation is not hindered by the air bubbles and a sufficient pressure rise is obtained.

According to this embodiment, since the fluid is supplied to the inlet channel 503 by the pump 700 at predetermined pressure, the fluid is supplied to the inlet channel 503 and the fluid chamber 501 even in a state in which the driving of the pulsation generator 100 is stopped. Therefore, it is possible to start an initial operation even if a priming water operation is not performed.

Since the fluid is ejected from the fluid-ejecting opening section 212 further reduced than the diameter of the outlet channel 511, fluid pressure is higher than the fluid pressure in the outlet channel 511. Therefore, it is possible eject the fluid at high speed.

Further, the fluid ejecting pipe 200 has rigidity enough for transmitting the pulsation of the fluid fed from the fluid chamber 501 to the fluid-ejecting opening section 212. Therefore, there is an effect that it is possible to eject a desired pulsed flow without hindering pressure propagation of the fluid from the pulsation generator 100.

Since the inertance of the inlet channel 503 is set larger than the inertance of the outlet channel 511, an increase in an outflow amount larger than a decrease in an inflow amount of the fluid to the fluid chamber 501 from the inlet channel 503 occurs in the outlet channel 511. Pulse-like fluid ejection into the fluid ejecting pipe 200 can be performed. Therefore, there is an effect that a check valve does not have to be provided on the inlet channel 503 side, the structure of the pulsation generator 100 can be simplified, cleaning of the inside is easy, and a concern about durability due to the use of the check valve can be eliminated.

Note that, if the volume of the fluid chamber 501 is suddenly reduced by setting the inertances of both of the inlet channel 503 and the outlet channel 511 sufficiently large, it is possible to suddenly increase the pressure in the fluid chamber 501.

By generating pulsation using the piezoelectric element 401 functioning as the volume varying unit and the diaphragm 400, it is possible to realize simplification of the structure of the pulsation generator 100 and a reduction in size involved in the simplification. A maximum frequency of a volume change of the fluid chamber 501 can be set to a high frequency equal to or higher than 1 KHz. This is optimum for ejecting of a high-speed pulsed flow.

The pulsation generator 100 generates a swirl flow in the fluid in the fluid chamber 501 with the inlet channel 503. Therefore, the pulsation generator 100 can push the fluid in the fluid chamber 501 in the outer circumferential direction of the fluid chamber 501 with a centrifugal force, concentrate the air bubbles included in the fluid on the center of the swirl flow, that is, in the vicinity of the axis of the substantial rotating body shape, and remove the air bubbles from the outlet channel 511 provided in the vicinity of the axis of the substantial rotating body shape. Consequently, it is possible to prevent a decrease in pressure amplitude due to the air bubbles held up in the fluid chamber 501 and continue stable driving of the pulsation generator 100.

Further, the inlet channel 503 is formed to communicate with the fluid chamber 501 through the route approaching the fluid chamber 501 while turning around the fluid chamber 501. Therefore, it is possible to generate the swirl flow without using a dedicated structure for swirling the fluid on the inside of the fluid chamber 501.

The groove-shaped inlet channel 503 is formed at the outer circumferential edge portion of the sealing surface 505 of the fluid chamber 501. Therefore, it is possible to form the inlet chamber 503 functioning as the swirl-flow generating unit without increasing the number of components.

Since the reinforcing plate 410 is provided on the upper surface of the diaphragm 400, the diaphragm 400 is driven with the opening section outer circumference of the reinforcing plate 410 as a fulcrum. Therefore, stress concentration less easily occurs. It is possible to improve the durability of the diaphragm 400.

Note that, if the corners of the joining surface of the reinforcing plate 410 to the diaphragm 400 are rounded, it is possible to further reduce the stress concentration of the diaphragm 400.

If the reinforcing plate 410 and the diaphragm 400 are stacked and integrally fixedly attached, it is possible to improve assemblability of the pulsation generator 100. Further, there is also a reinforcing effect of the outer circumferential edge portion of the diaphragm 400.

The fluid reservoir 507 for holding up the fluid is provided in the connecting section of the connection channel 504 and the inlet channel 503 on the inlet side to which the fluid is supplied from the pump 700. Therefore, it is possible to suppress the influence of the inertance of the connection channel 504 on the inlet channel 503.

Further, on the joining surface of the upper case 500 and the lower case 301, the ring-like packing 450 is provided in the position spaced apart in the outer circumferential direction of the diaphragm 400. Therefore, it is possible to prevent a leak of the fluid from the fluid chamber 501 and prevent a pressure drop in the fluid chamber 501.

### Preparation Operation During Abnormal Detection

As explained above, when the predetermined preparation operation (the preliminary pressurization, the priming processing, and the degassing processing) for the components 30a of the first group is completed and the pressure of the fluid in the fluid storing unit 765 is in the rough window, the fluid ejection device 1 according to this embodiment changes to a state in which the fluid can be ejected in a pulse-like manner from the first pulsating generating unit 100a. When the ON signal of the pulsation-generating-unit start switch 625 is input to the driving control unit 600 in this state, the first pump control unit 710a starts the supply of the fluid to the first pulsation generator 100a. On the other hand, the first driving-signal output unit 610a of the driving control unit 600 outputs a first drive signal to the first pulsation generator 100a and ejects the fluid from the first pulsation generator 100a in a pulse-like manner.

When malfunction occurs in the components 30a of the first group, the fluid ejection device 1 according to this embodiment can continue the ejecting of the fluid using the components 30b of the second group. In this way, by imparting redundancy to the components of the fluid ejection device 1, the more highly reliable fluid ejection device 1 is realized.

On the other hand, while malfunction does not occur in the components 30a of the first group, the fluid ejection device 1 according to this embodiment does not perform a preparation operation for the components 30b of the second group. When malfunction occurs in the components 30a of the first group, the fluid ejection device 1 starts the preparation operation for the components 30b of the second group. Consequently, it is possible to reduce wastes that occur when the redundancy is imparted to the components of the fluid ejection device 1.

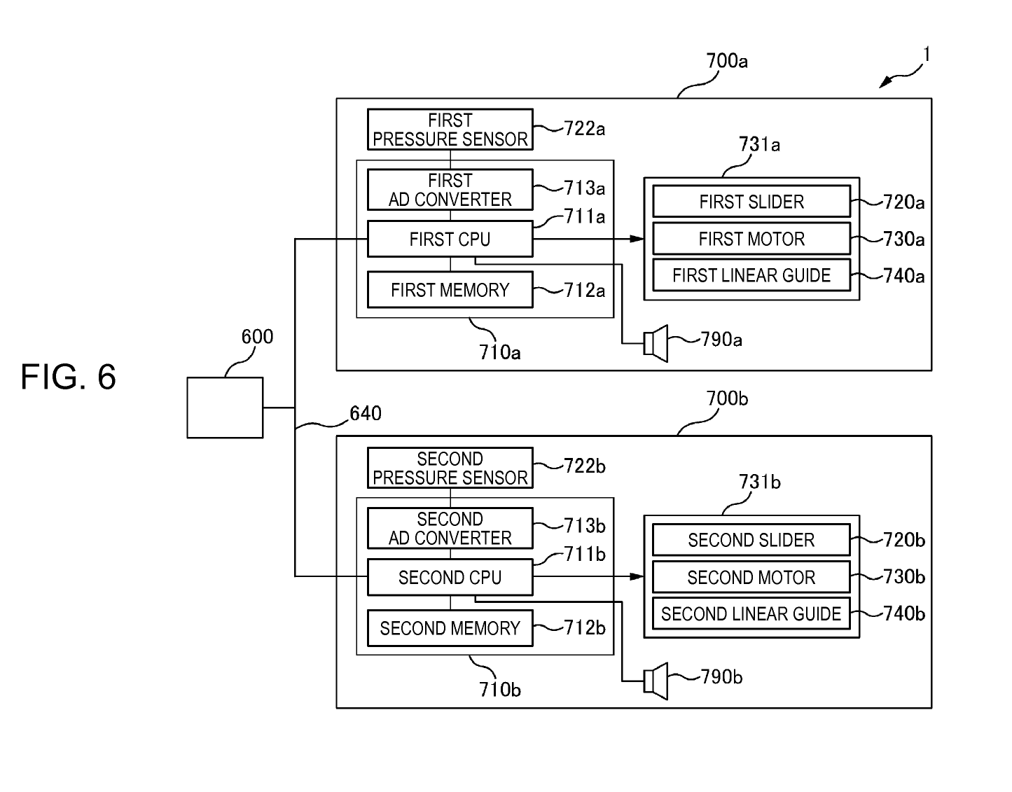

Content of control performed by the fluid ejection device 1 according to this embodiment is specifically explained below with reference to FIGS. 6 to 8. The fluid ejection device 1 performs the control in order to detect malfunction in the components 30a of the first group, start the preparation operation for the components 30b of the second group, and start ejecting of the fluid using the components 30b of the second group.

Note that, in the explanation in this embodiment, as an example, the first pump control unit 710a detects clogging that occurs in the first tube 25a because of, for example, mixing of foreign matters.

First, the configuration of the pump control unit 710 is explained with reference to FIG. 6 (the first pump control unit 710a and the second pump control unit 710b are collectively explained because the first pump control unit 710a and the second pump control unit 710b have the same configuration).

The pump control unit 710 includes a CPU (Central Processing Unit) 711, a memory 712, and an AD (Analog/Digital) converter 713.

The pump control unit 710 captures, from the pressure sensor 722, a detection signal of a level corresponding to pressure of the fluid pressing unit 731 in pressing the fluid storing unit 765 of the fluid container 760 and controls the fluid pressing section 731. For example, when the ON signal of the slider set switch 781 is input, the pump control unit 710 outputs a predetermined drive signal to the fluid pressing unit 731 to drive the motor 730 and controls the pressure to be within the rough window.

If the pressure is higher than a predetermined determination value, the pump control unit 710 determines that the connection tube 25 is clogged, outputs a stop signal explained below to the fluid pressing unit 731, and stops the pressing of the fluid storing unit 765. Note that the fluid pressing unit 731 includes the slider 720, the motor 730, and the linear guide 740.

The CPU 711 manages the control of the entire pump control unit 710. The CPU 711 executes a computer program configured from codes for performing various operations stored in the memory 712 to thereby realize the various functions according to this embodiment.

The memory 712 stores various data besides the computer program. For example, the memory 712 stores determination value level data indicating a level equivalent to the predetermined determination value.

The AD converter 713 receives an input of a detection signal output from the pressure sensor 722 and outputs data indicating a level of the detection signal. Specifically, the pressure sensor 722 detects pressure of the slider 720 in pressing the fluid storing unit 765 and outputs a detection signal of a level (e.g., a voltage) corresponding to the pressure. However, the AD converter 713 outputs detection level data (e.g., a voltage value) indicating the level of the detection signal output from the pressure sensor 722.

The CPU 711 captures the detection level data output from the AD converter 713 and compares the detection level data with the determination value level data stored in the memory 712.

If the detection level data is equal to or higher than the determination value level data, the CPU 711 outputs, to the fluid pressing unit 731, a stop signal for stopping the pressing of the fluid storing unit 765 by the fluid pressing unit 731.

The fluid pressing unit 731 immediately stops the driving of the motor 730 when the fluid pressing unit 731 receives the stop signal.

In this way, the fluid ejection device 1 according to this embodiment can detect the clogging of the connection tube 25 and prevent various deficiencies caused by the clogging. Consequently, it is possible to improve the safety and the reliability of the fluid ejection device 1.

When detecting the clogging of the connection tube 25 and outputting the stop signal, the CPU 711 may transmit a command for stopping the driving of the piezoelectric element 401 of the pulsation generator 100 to the driving control unit 600 and cause the pulsation generator 100 to stop the pulse-like ejecting of the fluid.

With such a form, it is possible to prevent high-pressure ejecting from being continuously performed from the pulsation generator 100 by the residual pressure in the connection tube 25. Therefore, it is possible to further improve the safety of the fluid ejection device 1.

When detecting the clogging of the connection tube 25 and outputting the stop signal, The CPU 711 may output a predetermined alarm. For example, the CPU 711 outputs, from a speaker 790, a message to the effect that the pressure in the connection tube 25 is higher than the determination value. Alternatively, the CPU 711 lights a predetermined alarm lamp (not shown in the figure).

With such a form, it is possible to quickly inform the operator such as the surgeon that the clogging has occurred in the connection tube 25. It is possible to further improve the safety of the fluid ejection device 1.

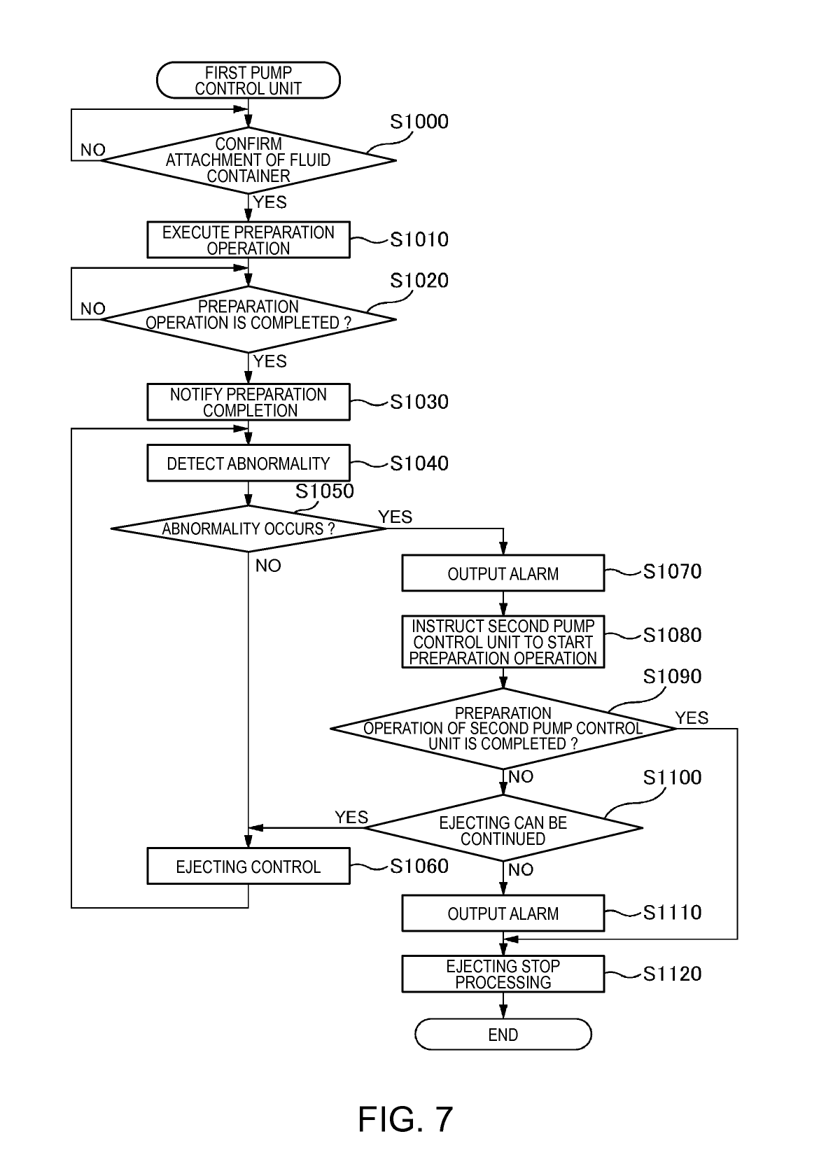

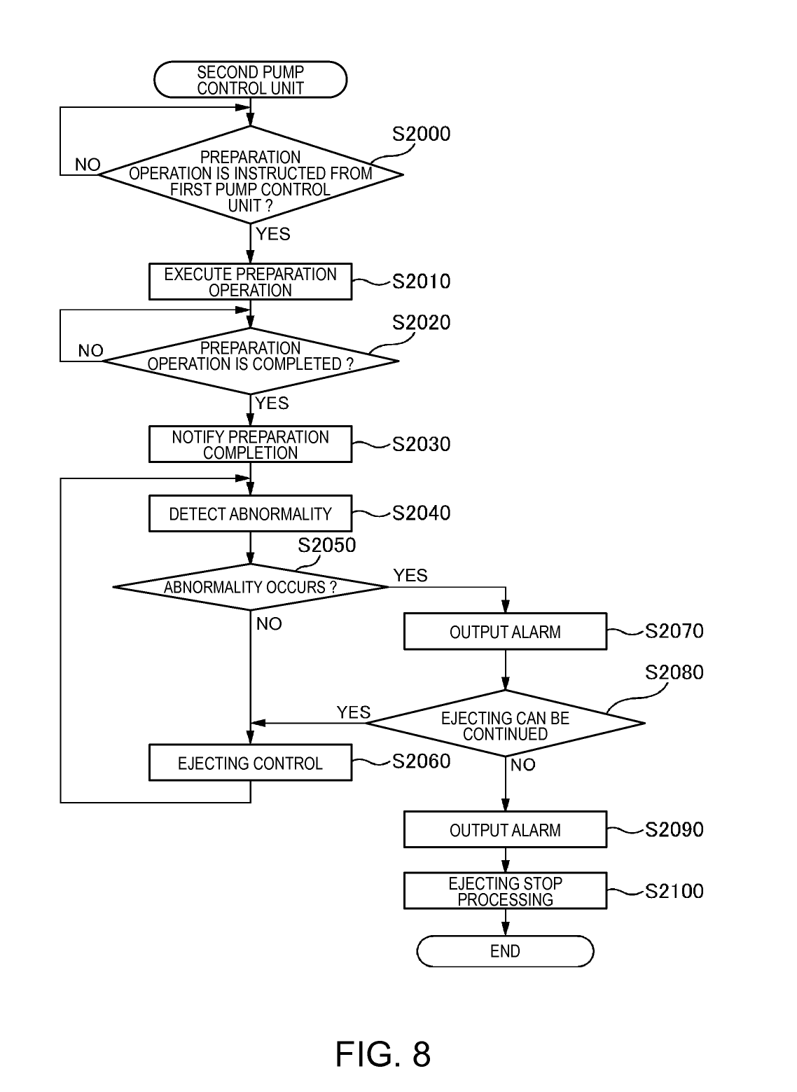

Respective control contents of the first pump control unit 710a and the second pump control unit 710b are explained with reference to flowcharts of FIGS. 7 and 8.

FIG. 7 is a flowchart for explaining the control content of the first pump control unit 710a. FIG. 8 is a flowchart for explaining control content of the second pump control unit 710b.

When a first fluid container 760a is attached to a first fluid-container attaching unit 770a (S1000), the first pump control unit 710a starts a preparation operation (S1010). As explained above, the first pump control unit 710a performs the preliminary pressurization, the priming processing, and the flushing processing as the preparation operation.

In this case, the first pump control unit 710a may individually perform the preliminary pressurization, the priming processing, and the flushing processing according to an instruction input from the operator. The first pump control unit 710a may perform the preliminary pressurization, the priming processing, and the flushing processing as a series of continuous processing even if the individual instruction input is not received from the operator.

When the preparation operation is completed (S1020), the first pump control unit 710a outputs notification information to the effect that the preparation operation is completed (S1030). For example, the first pump control unit 710a outputs, from a first speaker 790a, a sound message to the effect that the preparation operation is completed. Alternatively, the first pump control unit 710a lights a predetermined indicator lamp (not shown in the figure).

Subsequently, the first pump control unit 710a detects whether malfunction occurs in the components 30a of the first group (S1040). For example, the first pump control unit 710a detects whether the clogging occurs in the first connection tube 25a.

If malfunction does not occur, the first pump control unit 710a starts ejecting control (S1060). If the pressure of the fluid in a first fluid storing unit 765a is within the rough window, when being informed by the driving control unit 600 that the ON signal of the pulsation-generating-unit start switch 625 is input, as explained above, the first pump control unit 710a outputs the predetermined drive signal to a first fluid pressing unit 731a and starts the supply of the fluid to the first pulsation generator 100a.

While malfunction does not occur in the components 30a of the first group, the first pump control unit 710a continues the ejecting control (S1040, S1050, and S1060).

When detecting that malfunction occurs in the components 30a of the first group, the first pump control unit 710a outputs a predetermined alarm (S1070). For example, the first pump control unit 710a outputs, from the first speaker 790a, a sound message to the effect that the malfunction has occurred. Alternatively, the first pump control unit 710a lights a predetermined alarm lamp (not shown in the figure).

With such a form, it is possible to quickly inform the operator such as the surgeon that the malfunction has occurred in the components 30a of the first group. It is possible to further improve the safety of the fluid ejection device 1.

The first pump control unit 710a transmits, via a communication cable 640, a command for starting a preparation operation to the second pump control unit 710b (S1080). In response to the command, the second pump control unit 710b starts the preparation operation. Details of the preparation operation are explained below.

Thereafter, the first pump control unit 710a detects whether notification to the effect that the preparation operation of the second pump control unit 710b is completed is received via the communication cable 640 (S1090). The first pump control unit 710a determines, according to content of the malfunction that occurs in the components 30a of the first group, whether the ejecting can be continued or the ejecting should be stopped because of the malfunction (S1100).

When the content of the malfunction is, for example, the clogging of the first connection tube 25a, the first pump control unit 710a determines that the ejecting should be stopped. Alternatively, when the content of malfunction is, for example, occurrence of air bubbles in the first pulsation generator 100a, the first pump control unit 710a determines that the ejecting can be continued. Alternatively, when the content of the malfunction is movement of a first slider 720a to the residual amount position, the first pump control unit 710a determines that the ejecting can be continued.

When determining that the ejecting can be continued, the first pump control unit 710a continues the ejecting from the first pulsation generator 100a (S1060).

On the other hand, when determining that the ejecting should be stopped, the first pump control unit 710a outputs a predetermined alarm indicating to that effect (S1110). For example, the first pump control unit 710a outputs, from the first speaker 790a, a sound message to the effect that the ejecting is stopped. Alternatively, the first pump control unit 710a lights a predetermined alarm lamp (not shown in the figure).

With such a form, it is possible to quickly inform the operator such as the surgeon that the ejecting from the first pulsation generator 100a has been disabled. It is possible to further improve the safety of the fluid ejection device 1.

The first pump control unit 710a outputs a stop signal to the first fluid pressing unit 731a and instructs the driving control unit 600 via the communication cable 640 to stop the driving of the first pulsation generator 100a (S1120).

In S1090, even when receiving via the communication cable 640 notification to the effect that the preparation operation of the second pump control unit 710b is completed, in order to stop the ejecting of the fluid from the first pulsation generator 100a, the first pump control unit 710a outputs a stop signal to the first fluid pressing unit 731a and instructs the driving control unit 600 via the communication cable 640 to stop the driving of the first pulsation generator 100a (S1120).

Control content of the second pump control unit 710b is explained with reference to a flowchart of FIG. 8.

When receiving a command for a preparation operation start from the first pump control unit 710a (S2000), the second pump control unit 710b starts a preparation operation (S2010). The second pump control unit 710b performs the preliminary pressurization, the priming processing, and the flushing processing as the preparation operation. In this case, the second pump control unit 710b performs the preliminary pressurization, the priming processing, and the flushing processing as a series of continuous processing.

Note that, in this case, the pulsation-generating-unit changeover switch 629 is set by the operator to select the components 30a of the first group. However, the pulsation-generating-unit changeover switch 629 switches the fluid ejection device 1 to select the components 30b of the second group.

When the preparation operation is completed (S2020), the second pump control unit 710b outputs notification information to the effect that the preparation operation is completed (S2030). In this case, the second pump control unit 710b transmits, to the first pump control unit 710a via the communication cable 640, notification to the effect that the preparation operation is completed. The second pump control unit 710b outputs, from a second speaker 790b, a sound message to the effect that the preparation operation is completed. Alternatively, the second pump control unit 710b lights a predetermined indicator lamp (not shown in the figure). With such a form, it is possible to quickly inform the operator such as the surgeon that the ejecting from the second pulsation generator 100b has been enabled.

Subsequently, the second pump control unit 710b detects whether malfunction occurs in the components 30b of the second group (S2040). For example, the second pump control unit 710b detects whether the clogging occurs in the second connection tube 25b.

If malfunction does not occur, the second pump control unit 710b starts ejecting control (S2060). If the pressure of the fluid in a second fluid storing unit 765b is within the rough window, when being informed by the driving control unit 600 that the ON signal of the pulsation-generating-unit start switch 625 is input, as explained above, the second pump control unit 710b outputs the predetermined drive signal to a second fluid pressing unit 731b and starts the supply of the fluid to the second pulsation generator 100b.

While malfunction does not occur in the components 30b of the second group, the second pump control unit 710b continues the ejecting control (S2040, S2050, and S2060).

When detecting that malfunction occurs in the components 30b of the second group, the second pump control unit 710b outputs a predetermined alarm (S2070). For example, the second pump control unit 710b outputs, from the second speaker 790b, a sound message to the effect that the malfunction has occurred. Alternatively, the second pump control unit 710b lights a predetermined alarm lamp (not shown in the figure).

With such a form, it is possible to quickly inform the operator such as the surgeon that the malfunction has occurred in the components 30b of the second group. It is possible to further improve the safety of the fluid ejection device 1.

The second pump control unit 710b determines, according to content of the malfunction that occurs in the components 30b of the second group, whether the ejecting can be continued or the ejecting should be stopped because of the malfunction (S2080).

When the content of the malfunction is, for example, the clogging of the second connection tube 25b, the second pump control unit 710b determines that the ejecting should be stopped. Alternatively, when the content of malfunction is, for example, occurrence of air bubbles in the second pulsation generator 100b, the second pump control unit 710b determines that the ejecting can be continued. Alternatively, when the content of the malfunction is movement of a second slider 720b to the residual amount position, the second pump control unit 710b determines that the ejecting can be continued.

When determining that the ejecting can be continued, the second pump control unit 710b continues the ejecting from the second pulsation generator 100b (S2060).

On the other hand, when determining that the ejecting should be stopped, the second pump control unit 710b outputs a predetermined alarm indicating to that effect (S2090). For example, the second pump control unit 710b outputs, from the second speaker 790b, a sound message to the effect that the ejecting is stopped. Alternatively, the second pump control unit 710b lights a predetermined alarm lamp (not shown in the figure).

With such a form, it is possible to quickly inform the operator such as the surgeon that the ejecting from the second pulsation generator 100b has been disabled. It is possible to further improve the safety of the fluid ejection device 1.

The second pump control unit 710b outputs a stop signal to the second fluid pressing unit 731b and instructs the driving control unit 600 via the communication cable 640 to stop the driving of the second pulsation generator 100b (S2100).

The fluid ejection device 1 according to this embodiment is explained in detail above. With the fluid ejection device 1 according to this embodiment, it is possible to reduce wastes that occur when redundancy is imparted to the fluid ejection device 1.

Note that, in the embodiment, while the ejecting of the fluid is performed using the components 30b of the second group instead of the components 30a of the first group in which the malfunction is detected, when repairing of the components 30a of the first group is completed, it is also possible to put the components 30a of the first group on standby as backup and, in case where malfunction is detected in the components 30b of the second group, start the preparation operation of the components 30a of the first group.

With such a form, it is possible to further improve the reliability of the fluid ejection device 1.

In the explanation in the embodiment, the components 30a of the first group are used first to start the ejecting of the fluid. However, it is unnecessary to fixedly determine which of the components 30a of the first group and the components 30b of the second group are used first. It is also possible to freely select as appropriate according to a situation which of the components 30a of the first group and the components 30b of the second group are used first. In this case, when malfunction is detected in the selected the components 30a of the first group or components 30b of the second group, the fluid ejection device 1 starts the preparation operation of the other components 30a of the first group or components 30b of the second group. With such a form, it is possible to perform flexible operation of the fluid ejection device 1.

The preparation operation performed by the pump control unit 710 may be, for example, only the preliminary pressurization. In this case, for example, the operator can designate, by setting in the pump control unit 710, which preparation operation is performed. With such a configuration, it is possible to perform more flexible operation of the fluid ejection device 1.

The embodiment is intended to facilitate understanding of the invention and not limitedly interpret the invention. The invention could be modified and improved without departing from the spirit of the invention. Equivalents of the invention are also included in the invention.

What is claimed is:

1. A fluid ejection device comprising:
a first fluid ejection unit configured to eject fluid in a pulse-like manner according to a first drive signal;
a second fluid ejection unit configured to eject the fluid in a pulse-like manner according to a second drive signal;
a first fluid supplying unit configured to supply the fluid to the first fluid ejection unit via a first channel;
a second fluid supplying unit configured to supply the fluid to the second fluid ejection unit via a second channel;
a first fluid-ejecting control unit configured to execute a first preparation operation for changing the fluid in the first channel to a predetermined state and, after the first preparation operation is completed, output the first drive signal to the first fluid ejection unit;
a second fluid-ejecting control unit configured to execute a second preparation operation for changing the fluid in the second channel to a predetermined state and, after the second preparation operation is completed, output the second drive signal to the second fluid ejection unit; and
an malfunction detecting unit configured to detect malfunction that occurs in at least anyone of the first fluid ejection unit, the first fluid supplying unit, the first channel, and the first fluid-ejecting control unit, wherein
when the malfunction detecting unit detects the malfunction, the second fluid-ejecting control unit starts the second preparation operation.

2. The fluid ejection device according to claim 1, wherein
the malfunction detecting unit determines, when detecting the malfunction, according to content of the detected malfunction, whether the ejecting of the fluid by the first fluid ejection unit can be continued, and
the second fluid ejecting-control unit starts the second preparation operation even when it is determined that the ejecting of the fluid by the first fluid ejection unit can be continued.

3. The fluid ejection device according to claim 2, wherein, when determining that the ejecting of the fluid by the first fluid ejection unit cannot be continued, the malfunction detecting unit outputs predetermined notification information to that effect.

4. The fluid ejection device according to claim 1, wherein, when the second preparation operation is completed, the second fluid-ejecting control unit outputs predetermined notification information to that effect.

5. The fluid ejection device according to claim 1, wherein
the first preparation operation includes an operation for causing the fluid to reach the first fluid ejection unit from the first fluid supplying unit via the first channel, and
the second preparation operation includes an operation for causing the fluid to reach the second fluid ejection unit from the second fluid supplying unit via the second channel.

6. The fluid ejection device according to claim 5, further comprising:
a first pressure detecting unit configured to detect pressure of the fluid in the first channel; and
a second pressure detecting unit configured to detect pressure of the fluid in the second channel, wherein
the first preparation operation further includes an operation for causing the first fluid supplying unit to increase the pressure of the fluid in the first channel to a first predetermined value or more, and
the second preparation operation further includes an operation for causing the second fluid supplying unit to increase the pressure of the fluid in the second channel to a second predetermined value or more.

7. The fluid ejection device according to claim 6, wherein
the first preparation operation further includes an operation for causing the first fluid supplying unit and the first fluid ejection unit to remove air bubbles included in the fluid in the first channel, and
the second preparation operation further includes an operation for causing the second fluid supplying unit and the second fluid ejection unit to remove air bubbles included in the fluid in the second channel.

8. The fluid ejection device according to claim 7, wherein
the operation for causing the first fluid supplying unit and the first fluid ejection unit to remove the air bubbles included in the fluid in the first channel includes an operation for causing the first fluid ejection unit to eject the fluid in a pulse-like manner in a state in which the first fluid supplying unit is caused to increase a flow rate of the fluid flowing in the first channel in a predetermined time to a predetermined amount, and
the operation for causing the second fluid supplying unit and the second fluid ejection unit to remove the air bubbles included in the fluid in the second channel includes an operation for causing the second fluid ejection unit to eject the fluid in a pulse-like manner in a state in which the second fluid supplying unit is caused to increase a flow rate of the fluid flowing in the second channel in a predetermined time to a predetermined amount.

9. The fluid ejection device according to claim 1, further comprising:
a first pressure detecting unit configured to detect pressure of the fluid in the first channel; and
a second pressure detecting unit configured to detect pressure of the fluid in the second channel, wherein
the first preparation operation includes only an operation for causing the first fluid supply unit to increase the pressure of the fluid in the first channel to a first predetermined value or more, and
the second preparation operation includes only an operation for causing the second fluid supplying unit to increase the pressure of the fluid in the second channel to a second predetermined value or more.

10. The fluid ejection device according to claim 1, wherein, when detecting the malfunction, the malfunction detecting unit outputs predetermined notification information to that effect.
